(19) United States
(12) Patent Application Publication
(10) Pub. No.: US 2025/0041084 A1
(43) Pub. Date: Feb. 6, 2025

(54) Implant For Treating Aneurysms In The Area Of Bifurcations

(71) Applicant: phenox GmbH, Bochum (DE)

(72) Inventors: Hermann MONSTADT, Bochum (DE); Ralf HANNES, Dortmund (DE); Hans HENKES, Stuttgart (DE); Volker TRÖSKEN, Witten (DE)

(73) Assignee: phenox GmbH, Bochum (DE)

(21) Appl. No.: 18/717,189

(22) PCT Filed: Nov. 29, 2022
(86) PCT No.: PCT/EP2022/083690

§ 371 Date: Jun. 6, 2024

(30) Foreign Application Priority Data
Dec. 10, 2021 (DE) 10 2021 132 725.3

(51) Int. Cl. A61F 2/856; A61F 2/82; A61F 2/86

(52) U.S. Cl. CPC A61F 2/856; A61F 2/86; A61F 2002/823; A61F 2240/001

(57) ABSTRACT

An implant (1) for influencing the blood flow in the area of aneurysms which are localized at vascular branches of blood vessels. The implant is present in an expanded state, in which it is implanted in the blood vessel, and in a diameter-reduced, stretched state, in which it is movable through the blood vessel or a catheter. The implant (1) has at least three sections (2, 3) which, at least in the expanded state, are tubular and the walls of which are composed of interwoven wires (4) or interconnected struts, with at least two tubular sections (3) branching off from a first tubular section (2). The at least three tubular sections (2, 3) are made up individually and connected to one another at one end in each case in such a way that, in the expanded state, the blood flow through the first tubular section (2) into the tubular sections (3) branching off from the first tubular section (2) is ensured.

The invention relates to an implant for exerting an influence on the blood flow in the area of aneurysms that are localized at vessel branches. Aneurysms of this type are also referred to as bifurcation aneurysms.

Aneurysms are usually saclike or fusiform dilatations of the vessel wall primarily occurring in structurally weakened vessel wall areas due to blood pressure constantly acting on the wall. Accordingly, the inner vessel walls of an aneurysm are thus sensitive and susceptible to injury. Rupture of an aneurysm usually leads to significant health impairment, and in the case of cerebral aneurysms causes neurological deficits and even fatalities of patients.

Aside from surgical interventions, in which, for example, the aneurysm is clamped off by means of a clip, endovascular methods for the treatment of aneurysms are known in particular, with two approaches being primarily pursued. On the one hand, the aneurysm can be filled with occlusion agents, in particular so-called coils (platinum spirals). Coils facilitate the formation of thrombi and thus ensure occlusion of the aneurysm. On the other hand, it is known to close off the access to the aneurysm, for example the neck of an aciniform aneurysm, from the blood vessel side making use of stent-like implants and in this manner disconnect the aneurysm from the blood flow. Both methods serve to reduce the blood flow into the aneurysm and in this way alleviate, ideally even eliminating the pressure acting on the aneurysm so that the risk of a rupturing aneurysm is reduced.

When filling an aneurysm with coils it may happen that filling the aneurysm is inadequate, allowing blood flow into the aneurysm to continue resulting in the pressure acting on its inner wall to be maintained. The risk of the aneurysm steadily expanding and eventually rupturing still exists, albeit in an attenuated form. Moreover, this treatment method is primarily suitable for aneurysms with a relatively narrow neck-so-called aciniform aneurysms—as otherwise there is a risk that coils will protrude from a wide aneurysm neck into the blood vessel and thrombogenize there which may lead to occlusions in the vessel. In the worst case, a coil is completely washed out of the aneurysm resulting in vessels to be occluded elsewhere. To keep the coils in place in the aneurysm sac, the aneurysm neck is often additionally covered with a special stent.

Another intravascular treatment approach focusses on so-called flow diverters. These implants are similar in appearance to stents that are used for the treatment of stenoses. However, since the purpose of these flow diverters is not to keep a vessel open, but to obstruct access to the aneurysm on the blood vessel side, their mesh width is very narrow; alternatively, implants of this kind are coated with a membrane. Disadvantage of these implants is the risk that outgoing side branches in the immediate vicinity of the aneurysm to be treated may also be covered and thus closed off in the medium or long term.

Vascular branching, especially vascular bifurcation, is a quite frequently occurring phenomenon. In the event of a weak vessel wall, the blood stream flowing through an artery and acting on the front wall in a bifurcation quickly causes a protuberance or bulge which then rapidly dilates further. More often than not, such bifurcation aneurysms have a wide neck which makes therapy difficult to be performed with occlusion coils only.

Vascular implants that are suitable to bring about such a “latticing” of the aneurysm entrance in the area of a vascular branching are disclosed, for example, in international patent applications WO 2012/113554 A1 or WO 2014/029835 A1. By means of occlusion coils inserted after the implant has been placed in position the aneurysm can then be rendered nonhazardous. It is also possible that the implant itself decouples the aneurysm sufficiently from the blood flow. For this purpose, for example, the implant may have a membrane placed in the area of the aneurysm neck or in front of the aneurysm neck. If considered useful or expedient, the blood flow to the aneurysm can also be reduced with filaments alone, typically wires of small diameter, or membranes to such an extent that an additional introduction of occlusion coils or other occlusion means into the aneurysm can be dispensed with.

Publication WO 2018/134097 A1 describes a flow diverter system for treating bifurcation aneurysms as well as an insertion system relating thereto. In its expanded state, the flow diverter or bifurcation stent has three tubular sections that form the arms of the flow diverter, with two sections thereof intended for placement in branching vessels that fork off from a first section that is placed in the parent blood vessel supplying the blood. In this way a Y-structure is formed.

However, particularly in the case of flow diverters having three arms made up of interwoven wires, it was found to be problematic that the wires forming the braids are of different lengths in the area where the arms are connected to each other. When such an implant is brought to a compressed, stretched state in order to insert it into a catheter, the braided structure is thus destroyed particularly in the area where the three arms are connected. For that reason, a uniform surface coverage cannot be obtained after re-expansion, especially not in the area where the aneurysm is supposed to be covered and isolated from the flow of blood.

Based on the prior art described hereinbefore, the objective of the present invention therefore is to provide an implant which can be used to treat bifurcation aneurysms, said implant being easily brought into a compressed, stretched form for insertion through a catheter and by means of which, after expansion at the target position, uniform surface coverage can be achieved, especially in the region of the aneurysm neck.

As proposed by the invention, this objective is accomplished by providing an implant for influencing the blood flow in the area of aneurysms which are localized at vascular branches of blood vessels, wherein the implant is present in an expanded state, in which it is implanted in the blood vessel, and in a diameter-reduced, stretched state, in which it is movable through the blood vessel or a catheter, wherein the implant has at least three sections which, at least in the expanded state, are tubular and the walls of which are composed of interwoven wires or interconnected struts, with at least two tubular sections branching off from a first tubular section, the at least three tubular sections being made up individually and being connected to one another at one end in each case in such a way that, in the expanded state, the blood flow through the first tubular section into the tubular sections branching off from the first tubular section is ensured.

The invention is based on the findings that the properties of the implant as a whole can be improved if the tubular sections serving as arms of the implant are initially produced separately and then joined together at one end of each arm, resulting in an implant in which at least two further tubular sections branch off from a first tubular section. In most cases, the implant is composed of a total of three tubular sections, each of which forms an arm of the implant. By three tubular sections being initially formed individually it is ensured that these can be easily converted into a reduced-diameter, stretched state and an expanded state without destroying the braided structure in the area where the tubular sections adjoin each other. It is thus provided that a connection between the tubular sections is only created subsequently, which results in the connection being designed in such a way that the structure in the area of the connection is not negatively affected by expansion or compression of the implant. This means that the purely tubular sections can undergo expansion or compression without any problems, whereas the area in which the sections branch off from each other is not negatively affected by this, but instead the wires or struts can bring about a targeted, ideally pre-definable surface coverage here, as it is desired for the respective application. In this respect, the area of the bifurcation is of particular importance as this is where the neck of the aneurysm is usually located. The bifurcation implant is normally placed in such a way that a first tubular section is arranged in the parent blood vessel and the tubular sections branching off from it are located in the branching blood vessels, with the aneurysm having usually formed between the two branching blood vessels, so that the area of the implant in front of the aneurysm is precisely where two tubular sections branch off from the first tubular section.

In this context, “branching off” shall be understood to denote any transition from one tubular section to another tubular section. Branching off can be direct or indirect, that is, a tubular section can be directly adjacent to another tubular section or an intermediate section can be arranged between the two tubular sections, particularly a central connecting element as described hereinafter. Of importance in the context of the branching location is only that there is ultimately a transition from a first tubular section to further tubular sections, even if an intermediate section is still located between the tubular sections. In the expanded state, it must be ensured that the blood flow is maintained through the first tubular section and into the branching tubular sections.

The implant proposed by the invention is suitable to largely or completely decouple the bifurcation aneurysm, which is located at the branching location between the tubular sections, from the blood flow, due to the fact that at least some of the wires or struts come to lie in front of the aneurysm neck. It is to be noted that the flow of blood through the parent blood vessel, in which the first tubular section is arranged, and the branching blood vessels, in which the other two tubular sections are located, remains practically unaffected. As a result, the aneurysm becomes atrophic due to the lack of blood movement in the aneurysm allowing a thrombus to form and block the aneurysm.

Pursuant to an advantageous embodiment of the invention, the at least three tubular sections converge at a central connecting element, by means of which the tubular sections are connected to one another. As already mentioned, there are usually three tubular sections in total, i.e. a first tubular section arranged in the parent blood vessel and two additional tubular sections for the branching blood vessels. However, the individual tubular sections are not joined directly to each other, instead they are connected indirectly via the central connecting element, which typically has a structure different from that of the tubular sections themselves. In particular, the connecting element can be a laser-cut structure, whereas the tubular sections can be designed to consist of a braided structure of interwoven wires. Nevertheless, the tubular sections may also be laser-cut and correspondently consist of interconnected struts. The properties of the central connecting element, which is typically arranged directly in front of the aneurysm neck, can be tailored as required without directly interfering with the properties of the tubular sections.

The connecting element can be made up of a multitude of struts connected to each other. Typically, such a structure is produced using a laser cutting technique. The connecting element may expediently have a Y-structure, with each arm of the Y-structure being connected to one of the tubular sections. Accordingly, the connecting element has three arms, each of which merging into a tubular section. Therefore, the structure of the connecting element is adapted to the structure of the implant as a whole, which as a rule also forms a Y-structure.

Expediently, the interconnected struts of the connecting element are of identical length in the reduced-diameter, stretched state of the implant. This ensures that during expansion/compression no undesirable deformation of the area of the implant occurs that is crucial for an effective covering of the aneurysm neck.

The connection between the respective tubular section and the connecting element can be brought about in different ways. One possibility would be to secure or attach the tubular sections to the connecting element by means of wires or threads, typically one section on each arm of the Y-structure. Another option consists of creating a fusion bonded or material-to-material joint between the tubular sections and the connecting element or one arm of the Y-structure in each case. Fusion bonded joints include in particular welding, but other integral bonding methods such as adhesion or soldering are also conceivable in principle.

It is considered advantageous if the connecting element has a laser-cut structure. As regards implants such as flow diverters or stents, a distinction is usually made between braided structures composed of individual wires and laser-cut structures. However, other methods of manufacturing the implant are basically also conceivable, such as additive processes by means of which a structure is built up by adding material in a gradual manner. Such an additive method is in particular 3D printing.

The connecting element can be provided with one or more membranes covering the connecting element at least partially, which are arranged to completely or partially prevent the exit and entry of blood from or into the connecting element. In this way, a particularly effective coverage of the aneurysm neck is obtained so that the aneurysm is largely or completely isolated from the blood flow with the result that the risk of aneurysm rupturing is eliminated and the aneurysm atrophied.

Covering by a membrane shall be understood to mean any type of cover, that is, the membrane may be applied to the outside of the connecting element, attached to the inside of the connecting element, or the struts or wires of the connecting element may be embedded in the membrane.

Instead of using a central connecting element, it is also possible to connect the at least three tubular sections directly to each other by means of wires and/or threads. Another option is to attach the at least three tubular sections to each other by means of a fusion/material-to-material bond, with fusion bond in particular being understood to refer to welding. In addition, other material-to-material attachment methods such as the above-mentioned soldering or bonding are also conceivable in principle.

In this embodiment of the invention, the central connecting element has thus been dispensed with and the tubular sections abut directly as arms of the implant and are connected to each other. Nevertheless, the separate configuration of each individual tubular section ensures that expansion and compression of the tubular sections can take place without exerting a negative influence on the structure in the transitional area between the individual tubular sections.

In the event that wires and/or threads are used to join the tubular sections to each other or the tubular sections to the connecting element, these can be rendered at least partially radiopaque in order to allow visualization by the attending physician. In particular, wires made of a radiopaque material such as platinum, platinum-iridium or similar radiopaque metals and alloys can be employed.

The wires that connect the tubular sections to each other may be separate wires that are applied in addition to the tubular sections. Another option is to create the connection by means of wires that are part of the tubular sections themselves or emerge from them. These wires are linked or braided together to establish a connection between the tubular sections.

The tubular sections as well may have one or more membranes that at least partially cover the sections. In this manner, the flow of blood through the wall of the tubular sections is partially or completely obstructed. This can provide further assistance in isolating the aneurysm from the blood flow, especially if membranes are positioned near the branching point between the tubular sections. It is therefore also possible to provide areas of the tubular sections that are located close to the branching point with membranes, but not areas that are positioned further away from the branching point.

With respect to the membranes, what has been said above regarding membranes for the connecting element applies here as well, that is, a covering provided with the help of a membrane shall be understood to mean any type of coverage. The membrane can be applied to the outside of the tubular section or arranged on the inside of the tubular section, or the wires or struts of the tubular section are embedded in the membrane.

Both in the area of the connecting element and for the tubular sections, a membrane can be used that extends over larger areas, or several membranes may be provided that cover different areas. An option may also be to introduce radiopaque substances into the membranes. These may be radiopaque particles as they are customarily employed as contrast medium for radiotechnological purposes. Such radiopaque substances are, for example, heavy metal salts such as barium sulfate or iodine compounds. A radiopaque membrane proves beneficial during placement of the implant and for localization purposes and may be used either additionally to or instead of marker elements.

Membranes may also be designed to have an antithrombogenic effect or an effect that promotes endothelial formation. Such an effect is particularly desirable where the implant is adjacent to normal vessel walls because blood flow through the vessels should not be impaired and, moreover, good anchorage of the implant in the vascular system should be achieved. Membranes can possess the desired characteristics by themselves through an appropriate choice of material, but they can also be provided with coatings that produce the desired effects.

Within the meaning of the present invention, a membrane is a thin structure having a planar surface, regardless of whether said structure is permeable, impermeable or partially permeable to liquids. However, to accomplish the objective of the aneurysm treatment, membranes are preferred that are completely or at least substantially impermeable to fluids such as blood. In addition, a membrane may also be provided with pores, particularly in the region of the aneurysm neck, through which occlusion agents can be introduced into the aneurysm. Another option is to have the membrane designed in such a way that it can be pierced with a microcatheter for the introduction of occlusion agents or even with the occlusion agents themselves.

The membranes can be made of polymer fibers or polymer films. Preferably, the membranes are produced by an electrospinning process. In this process, the wires/struts are normally embedded in the membrane. This can be achieved by spinning or braiding the wires/struts with fibers.

In electrospinning, fibrils or fibers from a polymer solution are deposited on a substrate with the help of an electric current. Said deposition causes the fibrils to agglutinate into a non-woven fabric. Usually, the fibrils have a diameter ranging between 100 and 3000 nm. Membranes created by electrospinning have a very uniform texture. The membrane is tenacious, withstands mechanical stresses, and can be pierced mechanically without an opening so created giving rise to cracks propagating from it. The thickness of the fibrils as well as the degree of porosity can be controlled by selecting process parameters as appropriate. In the context of producing the membrane and with respect to materials suitable for this purpose, special attention is drawn to publications WO 2008/049386 A1, DE 28 06 030 A1, and literature referred to therein.

In lieu of electrospinning, the membranes may also be produced by an immersion or spraying process such as spray coating. With respect to the material used for the membranes, it is important that they are not damaged by the mechanical stresses arising during implant insertion into the blood vessel system. To ensure this, the membranes should have sufficient elasticity.

The membranes can be made of a polymer material such as polytetrafluoroethylene, polyester, polyamides, polyurethanes, polyolefins or polysulfones. Especially preferred are polycarbonate urethanes (PCU). In particular, an integral connection between the membranes and the wires/struts is desirable. Such an integral connection can be achieved by covalent bonds provided between the membranes and the wires/struts. The formation of covalent bonds is promoted by silanization of the wires/struts, that is, by a chemical bonding of silicon compounds, in particular silane compounds, to at least portions of the surface of the wires/struts. On surfaces, silicon and silane compounds attach, for example, to hydroxy and carboxy groups. Basically, aside from silanization, other methods of mediating adhesion between the wires/struts and the membranes are also conceivable.

Silane compounds in this context are to be seen as all those compounds which follow the general formula RₘSiXₙ (m, n=0-4), where R stands for organic radicals, in particular alkyl, alkenyl or aryl groups, and X stands for hydrolyzable groups, in particular OR, OH or halogen, with R=alkyl, alkenyl or aryl. In particular, the silane may have the general formula RSiX₃. Moreover, relevant compounds having several silicon atoms also count among the silane compounds. In particular, silane derivatives in the form of organosilicon compounds are regarded as silane compounds in this context.

As already mentioned hereinbefore, additional substances promoting endothelial formation may be embedded in or deposited on the membranes or wires/struts. Because aneurysms arise due to degenerative diseases of the vascular wall, the promotion of endothelial formation and correction of endothelial dysfunction may yield beneficial effects. This applies especially to the area where the aneurysm is in contact with the flow of blood in the respective blood vessel (parent vessel). Preferably, substances promoting endothelial formation are applied to the outside of the membrane, with outer side being understood here to denote the side of a membrane facing the vessel wall in the implanted state and the inner side being understood to mean the membrane side facing away from the vessel wall. Hyaluronic acid, statins (3-hydroxy-3-methylglutaryl coenzyme A reductase inhibitors), and other polymers may promote endothelial cell colonization. Polysaccharides, especially glycosaminoglycans, which are able to mimic the glycocalyx, are particularly suitable polymers. Another material that can be used is POSS-PCU (polyhedral oligomeric silsesquioxane poly(carbonate-urea) urethane). It is a nanocomposite that has been described inter alia as a scaffold for artificial organs and as a coating for medical devices (Tan et al., Crit Rev. Biomed Eng. 2013; 41 (6): 495-513). It is also possible to use POSS-PCL (polyhedral oligomeric silsesquioxane poly(caprolactone-urea) urethane). It applies to both POSS-PCU and POSS-PCL that in particular functionalized derivatives of these nanocomposites can also be employed. This is especially true for those derivatives that can be obtained by linking with polyacrylic acid (poly-AA). Since POSS-PCU and/or POSS-PCL nanocomposite polymers are only poorly suited for direct immobilization on the surface of an implant, it has been found advantageous to combine polymers such as polyacrylic acid (poly-AA) with the nanocomposite. This can be achieved, for example, by plasma polymerization of acrylic acid. A poly-AA-g-POSS-PCU surface obtained in this way promotes the bonding of collagen (especially collagen type 1) and thus endothelial formation (see Solouk et al., Mater Sci Eng C Mater Biol Appl. 2015; 46:400-408). In general, biofunctional or bioactive coatings may be present on the membrane.

The at least two tubular sections branching off from the first tubular section may have a smaller diameter in the expanded state than the first tubular section. This is, on the one hand, due to the fact that the branching blood vessels frequently exhibit a smaller inner diameter than the parent blood vessel. On the other hand, the smaller diameter of the branching tubular sections ensures that stress-free connection is achieved with the first tubular section.

The walls of the tubular sections can be formed from interwoven wires. In this context, it is also conceivable to braid at least some of the tubular sections in such a way that no free wire ends exist on at least one end of the respective tubular section. Therefore, the tubular section at this end is braided closed in relation to the circumference of the wall. This means that interwoven wires do not end freely here, but are guided back into the braiding in the opposite direction.

The tubular sections, for example, can be designed in such a way that each tubular section features one end with free wire ends and one end without free wire ends. In other words, each tubular section is braided open at one end and braided closed at the other, with open and closed in this case not referring to the lumen but to the circumference of the tubular sections. The tubular sections in this case, preferably converge centrally with the ends that do not have free wire ends (i.e. that are braided closed). They can be connected there to a central connecting element or directly to the other tubular sections. In this context, the wires that compose the tubular sections can also be employed directly to create a connection with the connecting element or the other tubular sections, in particular by interlacing the wires with struts of the connecting element or wires of the other tubular sections.

As regards the braided tubular sections, the braiding, in principle, may be plaited in any known way. It may have a one-plaited and/or multi-plaited structure. Especially when used in a narrowly plaited configuration a dense braiding will cause the individual wires to be highly stressed. However, while a multi-plaited design is conducive to removing stresses from the braid, a too highly plaited arrangement on the other hand will cause the bond in the braid to deteriorate. The plaiting method indicates how many times a given wire passes crossing wires on the same side of such wires before it changes sides and subsequently passes on the other side of a corresponding number of crossing wires. In case of a two-plaited arrangement a wire, for example, passes in succession over two crossing wires and then in succession along the underside of two crossing wires. In a one-plaited structure the wires are arranged alternately one above the other and one below the other.

In particular, also multi-ply wires may be employed. The plying method indicates the number of joined, parallelly arranged individual wires. Single or multiple plying may be provided with one or several individual wires extending in parallel. Since during the braid manufacturing process wires are introduced into the process from bobbins, one or several individual wires are fed from the respective bobbin simultaneously to the mandrel on which the braiding is produced. Each wire may consist of a single wire or of strands comprising several individual wires joined and preferably twisted together.

A plying of two or an even higher plying configuration results in a higher surface density of the braiding and at the same time reduces the longitudinal expansion when the braiding is compressed. Such a higher surface density, however, causes flexibility to diminish, also through increased friction and tension. This may be counteracted by making use of a more highly plaited arrangement, i.e. a two-plaited or higher-plaited structure will result in higher flexibility.

The total number of wires forming the tubular sections is preferably 24 to 96, thus creating a dense braid structure that adapts well to the inner wall of the blood vessels. Aside from this, the high number of wires ensures good surface coverage and braid density in the area of the aneurysm.

Different forms of filaments can be used as wires or struts. These may have a round, oval or even angular cross-section, in particular a rectangular, square or trapezoidal cross-section, and in the case of an angular cross-section the edges can be rounded. Flat wires or struts in the form of thin strips may be employed as well. The individual wires can also be made up of several individual filaments that are twisted together or extending in parallel. The wires or struts can be solid or also hollow inside. Additionally, the wires/struts may be subjected to electropolishing to make them smoother and rounder and thus render them less traumatic. This also reduces the risk of germs or other impurities adhering.

Preferably, the wires or struts are made of metal, but in principle the use of wires made of other materials, for example plastics or polymer materials, is also conceivable. Auch solche Filamente werden erfindungsgemäβ als Drähte oder Stege verstanden.

To ensure that the implant, when liberated and released in the blood vessel, for example from or out of a catheter, automatically expands and adapts to the inner walls of the blood vessels, it is preferred to make the implant at least partially from a material having shape memory properties. Nickel-titanium alloys, for example nitinol, or ternary nickel-titanium-chromium alloys or nickel-titanium-copper alloys are particularly preferred in this context. However, other shape memory materials, for example other alloys or even shape memory polymers, are also conceivable. Materials having shape memory properties enable an implant to be imprinted with a secondary structure that it will automatically strive to adopt as soon as it is no longer hindered from expanding. The use of cobalt-chromium or cobalt-chromium-nickel alloys is also possible.

It is also possible to use so-called DFT® (drawn filled tubing) wires or struts, that is, wires/struts in which the core of the wire/strut is made of a different material than the sheathing surrounding the core. It is particularly expedient to use wires/struts having a core consisting of a radiopaque material and a sheathing of a material that has shape memory properties. The radiopaque material may, for example, be platinum, a platinum-iridium alloy, or tantalum, and the material having shape memory properties is preferably a nickel-titanium alloy, as mentioned above. DFT® filaments of this type are offered by Fort Wayne Metals, for example.

Respective wires or struts combine the advantageous characteristics of two materials. The sheathing having shape memory characteristics will ensure that the implant is allowed to expand and adapt to the vessel walls, whereas the radiopaque material ensures that the implant is visible on radiographs and can thus be monitored by the attending physician and positioned as needed.

The distal and proximal ends of the wires or struts are preferably configured with a view to precluding injury to the vessel walls. For example, wires/struts can be rounded at their ends and thus rendered atraumatic. Appropriate forming can be done by remelting with the help of a laser. It is also possible to join one or more wires/struts at each end and thus create appropriately shaped atraumatic terminations. It is, in particular, recommendable and expedient to avoid pointed wire ends.

In the event of laser-cut tubular sections or, expressed more generally, tubular sections composed of interconnected struts, both open-cell and closed-cell structures can be employed. Closed-cell structures can usually be repositioned more easily or allow the implant to be completely replaced during the intervention. Open-cell structures, on the other hand, adapt better to the routing of the vascular anatomy.

To further increase the surface coverage in the area of the aneurysm neck, the implant can undergo a shaping process during manufacture to increase the number of wires or struts at the branching location in the distal direction. This is of particular importance in embodiments in which no connecting element is arranged between the tubular sections. The bifurcation location pointing in distal direction typically corresponds to the neck of the aneurysm, so that increasing the number of wires/struts at this position provides greater coverage of the aneurysm neck and thus more effective isolation from the blood supply. The appropriate shaping processes can be of a mechanical nature, however, it is in particular at least also a heat treatment which ensures that the implant in its expanded state comprises a high number of wires or struts at the branching point in distal direction. Such a heat treatment is particularly expedient for wires or struts consisting of a shape memory material, because the heat treatment enables to imprint a structure on the wires and in this way also on the entire implant, which the implant strives to adopt when released in the vascular system.

In an expanded state of the implant, advantageous coverage rates at the branching site in the distal direction range between 25 and 75%, preferably between 35 and 65%.

Unless the context indicates otherwise, the term expanded state within the meaning of the invention is understood to denote a state which the implant assumes when it is not subject to any external constraints. Depending on the diameter of the blood vessels in which the implant is placed, the expanded state in the vasculature may differ from the expanded state existing in the absence of external constraints because the implant may not be able to assume its fully expanded state. In the completely expanded state, the tubular sections advantageously have an outer diameter ranging between 1.5 mm and 7 mm, said diameter can be adapted to the respective target site in the blood vessel system. More often than not, the first, proximally located tubular section has a larger outer diameter than the more distally positioned tubular sections due to the fact that the diameter of the parent blood vessel is larger than that of the branching blood vessels. The overall length of the implant in the expanded state is usually between 5 mm and 100 mm, and in particular between 10 and 50 mm when the implant is placed such that the two distally located tubular sections have a parallel configuration and extend in the same direction as the proximal tubular section. The wires or struts forming the implant can, for example, have a diameter or thickness ranging between 20 and 60 μm.

On the other hand, the implant may also be present in a diameter-reduced elongated state, which may also be referred to as a contracted or compressed state, with the terms being used synonymously in the context of the present invention in the sense that the implant resp. the tubular sections have a significantly smaller radial expansion in the diameter-reduced state than in the expanded state. A stretched, contracted/compressed state is assumed, for example, when the implant is brought to the target site with the help of a catheter. It is also possible to mount the implant on the outside of a catheter, tube or similar item, in which case the implant is also held in a less radially enlarged state as compared to its expanded state.

With respect to the implant placement process, the terms “proximal” and “distal” are to be understood such that they refer to parts of the implant that point towards the attending physician (proximal), or, as the case may be, to parts that point away from the attending physician (distal). Typically, the implant is thus moved forward in distal direction by or with the aid of a catheter. The term “axial” refers to the longitudinal axis of the implant extending from proximal to distal while the term “radial” refers to directions perpendicular to this.

To further improve the occlusion of the aneurysm, in addition to isolating the aneurysm from the flow of blood by the implant, occlusion agents may also be introduced into the aneurysm, for example coils as they are known in the prior art. Also possible is the insertion of viscous embolizates.

The implant proposed by the invention is usually provided with radiopaque marker elements facilitating visualization and positioning accuracy at the placement site. For example, such marker elements can be provided in the form of wire coils, sleeves and slotted tube sections, which are fixed to the implant. For said marker elements, in particular platinum and platinum alloy materials are suitable, for example an alloy of platinum and iridium, as it is frequently used according to the state of the art for marking purposes and as material for occlusion coils. Other usable radiopaque metals are tantalum, gold, and tungsten. Another option is to fill the wires or struts with a radiopaque material, as mentioned hereinbefore. It is also possible to provide the implant, in particular the wires or struts, with a coating consisting of a radiopaque material, for example applying a gold coating. This can have a thickness of 1 to 6 μm, for example. The radiopaque material coating need not be applied to the entire implant. Nevertheless, even when applying a radiopaque coating it is considered useful to arrange one or several radiopaque markers on the implant, in particular at the distal end of the implant.

The implant provided by the invention is particularly suitable for the treatment of intracranial bifurcation aneurysms, but its use for other types of aneurysms, for example aortic aneurysms or peripheral aneurysms, is also conceivable, in which case the dimensions of the implant are to be adapted as appropriate. As regards placing the implant in front of the bifurcation aneurysm, an insertion system can be used as described in publication WO 2018/134097 A1.

The invention also relates to a method of manufacturing an implant as described above. For this purpose, at least three (in most cases exactly three) tubular sections are initially manufactured individually. Each tubular section is made up of interconnected struts or interwoven wires. The tubular sections are each connected to one another at one end in such a way that a liquid can flow from a first tubular section into the tubular sections that branch off from this first tubular section. When put to the intended use, the liquid referred to is blood. The connection of the tubular sections can be directly or indirectly. In the event of an indirect connection, a central connecting element is arranged between the tubular sections. In the case of a direct connection, the connection can be made, for example, by providing a fusion bond, preferably by welding, or with the aid of wires or threads.

Aside from the implant and the method of manufacturing the implant, the invention also relates to the use of the implant for the treatment of arteriovenous malformations, in particular (bifurcation) aneurysms, as well as to combining the implant with an insertion system and, as the case may be, a microcatheter. All statements made with reference to the implant itself shall also apply in an analogous manner to the use of the implant, the method of manufacturing the implant and a method of application of the implant as well as the combination of the implant with an insertion system.

Further elucidation of the invention is provided by way of examples through the enclosed figures. It is to be noted that the figures show preferred embodiment variants of the invention, but with the invention itself not being limited thereto. To the extent it is technically expedient, the invention comprises, in particular, any optional combinations of the technical features that are stated in the claims or in the description as being relevant to the invention.

Clarification of the invention is provided by the following figures where

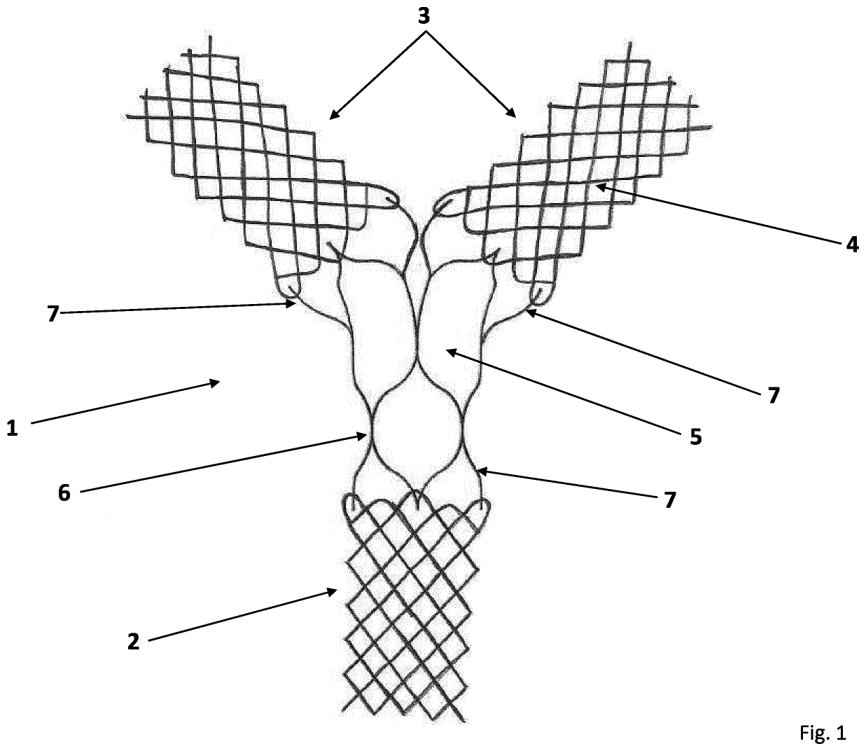

FIG. 1 shows an implant according to a first embodiment;

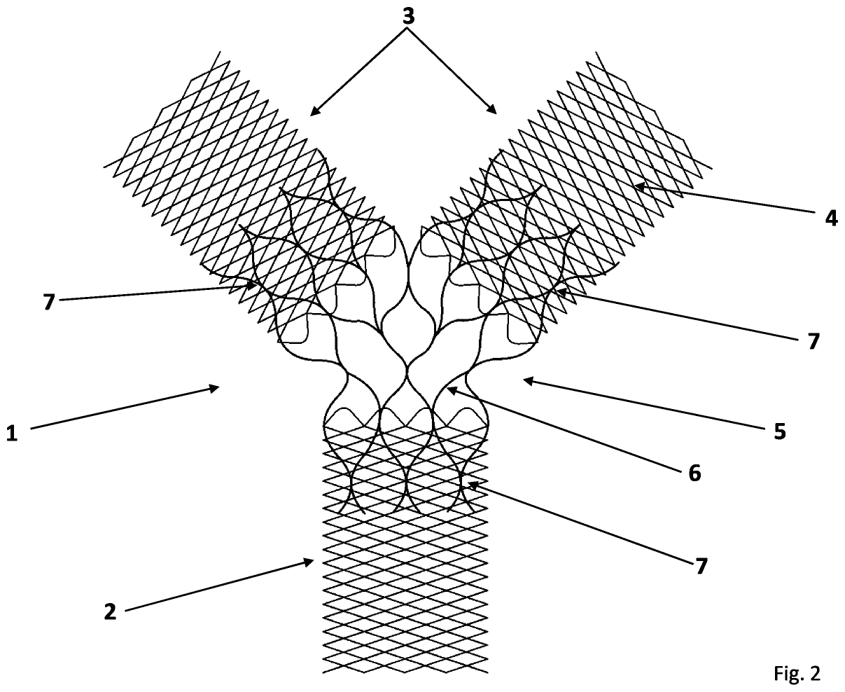

FIG. 2 is a modification of the implant according to the first embodiment;

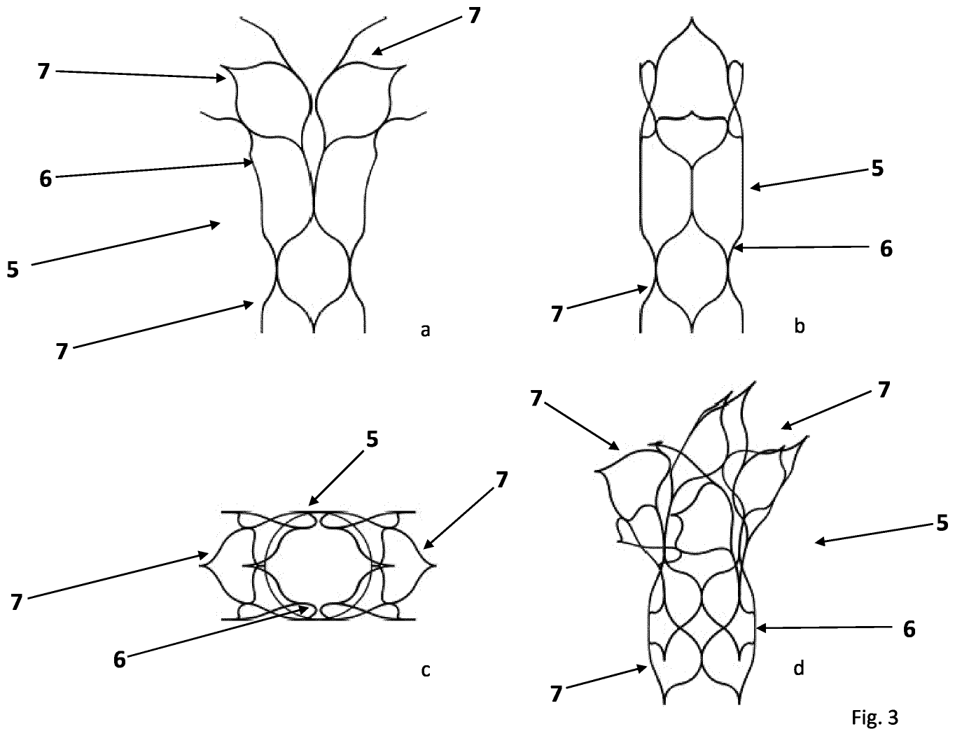

FIGS. 3a through 3d illustrates various views of a central connecting element;

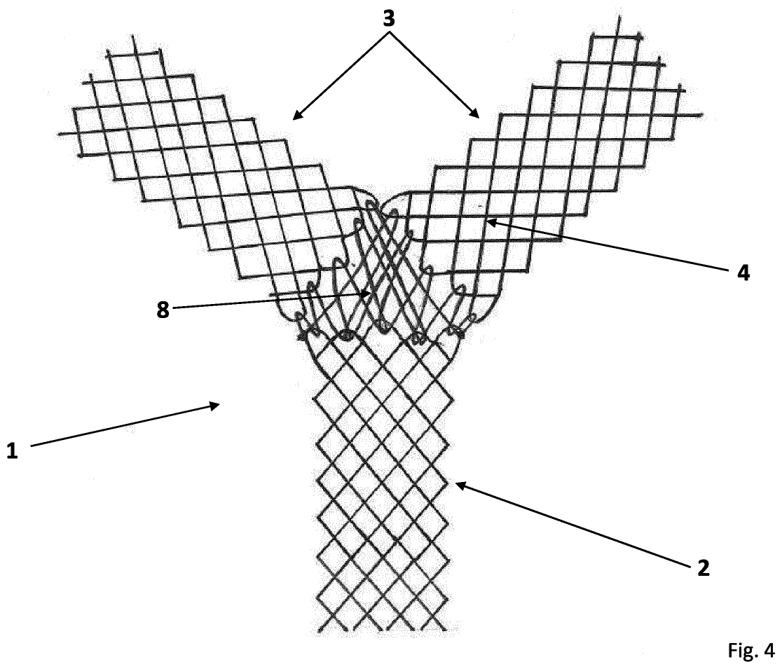

FIG. 4 shows an implant according to a second embodiment;

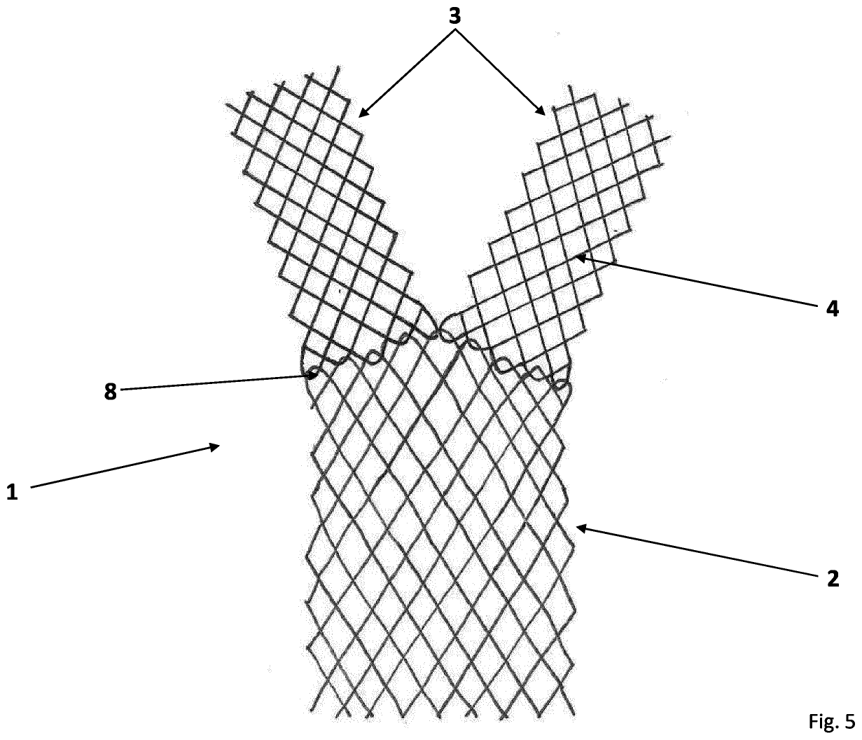

FIG. 5 shows an implant according to a third embodiment.

FIG. 1 is the illustration of the implant 1 according to a first embodiment. In the embodiment shown here, three tubular sections 2, 3 are each connected at one end to a central connecting element 5, which itself is present in the form of a Y-structure comprising three arms 7 provided for connection to the tubular sections 2, 3. The first tubular section 2 is placed in the parent blood vessel, while the other distal tubular sections 3 are located in the branching blood vessels. The tubular sections 2, 3 are formed from interwoven wires 4, with the central connecting element 5 being a laser-cut structure comprising individual struts 6. Each of the tubular sections 2, 3 are welded at one end to the central connecting element 5.

FIG. 2 shows a variant of the first embodiment, wherein a proximal tubular section 2 is also connected to two distally located tubular sections 3 by means of a central connecting element 5. Here the tubular sections 2, 3 are as well made up of individual wires 4 braided together, with the central connecting element 5 consisting of laser-cut struts 6. The central connecting element 5 forms a Y-structure with three arms 7, each of which being connected to a tubular section 2, 3. However, in comparison to the variant depicted in FIG. 1, the arms 7 of the Y-structure have a greater overlap with tubular sections 2, 3. In this case as well, the connection is brought about by welding the wires 4 to the laser-cut connecting element 5.

FIGS. 3a through 3d show various views of the central connecting element 5. As in FIGS. 1 and 2, FIG. 3a is a view from the side; in FIG. 3b the central connecting element 5 is rotated by 90°. FIG. 3c is a view seen from above and FIG. 3d illustrates an oblique view seen from the side. Again, the structure consisting of struts 6 can be observed, the connecting element 5 forming a total of three individual arms 7.

FIG. 4 is an illustration of a second embodiment of the invention, in which the implant 1 again comprises a first tubular section 2 for placement in the parent blood vessel as well as two more distal tubular sections 3 for placement in the branching blood vessels. Again, the tubular sections 2, 3 are braided structures consisting of individual wires 4; however, in contrast to the first embodiment no central connecting element 5 is provided in this case, instead the three tubular sections 2, 3 are connected to one another by means of wires 8, which are threaded through the end meshes of the tubular sections 2, 3.

A third embodiment of the invention is shown in FIG. 5, which, basically, is similar to the second embodiment, except that no additional wires 8 are provided in this embodiment, instead the wires 8 used to connect the tubular sections 2, 3 are themselves wires of the tubular sections 2, 3. In this case, the individual tubular sections 2, 3 are thus intertwined at the ends. Moreover, it can be seen that the tubular sections 3 located more distally have a smaller diameter than tubular section 2 arranged proximally, which is due to the fact that the inner diameter of the parent blood vessel is usually larger than that of the branching blood vessels.

1. An implant for influencing the blood flow in the area of aneurysms which are localized at vascular branches of blood vessels, wherein the implant is present in an expanded state, in which it is implanted in the blood vessel, and in a diameter-reduced, stretched state, in which it is movable through the blood vessel or a catheter, wherein the implant has at least three sections which, at least in the expanded state, are tubular and the walls of which are composed of interwoven wires or interconnected struts, with at least two tubular sections branching off from a first tubular section,
wherein
the at least three tubular sections being made up individually and being connected to one another at one end in each case in such a way that, in the expanded state, the blood flow through the first tubular section into the tubular sections branching off from the first tubular section is ensured.

2. The implant according to claim 1, wherein the at least three tubular sections are brought together at a central connecting element, by means of which said at least three tubular sections are connected to each other.

3. The implant according to claim 2, wherein the connecting element is composed of several interconnected struts.

4. The implant according to claim 2 wherein, the connecting element has a Y-structure, wherein each arm of the Y-structure being attached to one of the tubular sections.

5. The implant according to claim 2, wherein the interconnected struts of the connecting element are of identical length in the diameter-reduced, stretched state of the implant.

6. The implant according to claim 2, wherein the tubular sections are attached to the connecting element by means of wires or threads.

7. The implant according to claim 2, wherein the tubular sections are secured to the connecting element by means of a fusion or material-to-material bond.

8. The implant according to claim 2, wherein the connecting element is a laser-cut structure.

9. The implant according to claim 2 wherein the connecting element is provided with one or more membranes which at least partially cover the connecting element, said membranes completely or partially preventing the exit and entry of blood from or into the connecting element.

10. The implant according to claim 1 wherein, the at least three tubular sections are connected to each other by wires and/or or threads.

11. The implant according to claim 10 wherein, the wires and/or or threads have at least partially radiopaque characteristics.

12. The implant according to claim 10 wherein, the wires form part of the tubular sections or originate from them.

13. The implant according to claim 1, wherein the at least three tubular sections are connected to each other by a fusion or material-to-material bond.

14. The implant according to claim 1 wherein one or more membranes at least partially cover the tubular sections.

15. Implant according to claim 1 wherein the at least two tubular sections branching off from the first tubular section have a smaller diameter in the expanded state than the first tubular section.

16. Implant according to claim 1 wherein the walls of the tubular sections are composed of wires braided together, wherein at least some of the tubular sections being braided in such a way that at least one end of the respective tubular section has no free wire ends.

17. The implant according to claim 16, wherein each tubular section features one end with free wire ends and one end without free wire ends.

18. The implant according to claim 17, wherein the tubular sections with those ends that have no free wire ends come together at a central connecting element or are centrally connected to one another.

19. Method A method for the manufacture an implant in accordance with claim 1 wherein the at least three tubular sections are first manufactured individually and subsequently joined together at one end in each case.
